(19) United States
(12) Patent Application Publication
(10) Pub. No.: US 2022/0228195 A1
(43) Pub. Date: Jul. 21, 2022

(54) SAMPLE COLLECTION METHODS AND DEVICES

(71) Applicant: Viome Life Science, Inc., Bellevue, WA (US)

(72) Inventors: Momchilo VUYISICH, Bothell, WA (US); Andrew J. HATCH, Santa Fe, NM (US)

(21) Appl. No.: 17/616,650

(22) PCT Filed: Jun. 5, 2020
(86) PCT No.: PCT/US20/70125

§ 371 Date: Dec. 4, 2021

Related U.S. Application Data
(60) Provisional application No. 62/857,814, filed on Jun. 5, 2019.

(51) Int. Cl. C12Q 1/6806; C12N 15/10; B01L 3/00

(52) U.S. Cl. CPC C12Q 1/6806; C12N 15/1065; C12N 15/1096; C12N 15/1006; B01L 2300/021; B01L 3/5029; B01L 2200/0647; B01L 2200/185; B01L 2300/046; B01L 3/50825

(57) ABSTRACT

A kit comprises: (i) one or a plurality of collection containers, each comprising a cap and, attached to the cap, an applicator, wherein the applicator is configured to fit within the collection container when closed by the cap; (ii) an aqueous fluid comprising a nucleic acid preservative; and (iii) solid particles adapted for lysing cells, such as bacteria, by bead beating. Upon receipt in a facility, the contains are placed onto a bead beating instrument without aliquoting samples from the collection container into another in bead beating container, or adding solid particles for bead beating.

### REFERENCE TO RELATED APPLICATIONS

This application claims the benefit of the priority date of U.S. provisional application 62/857,814, filed Jun. 5, 2019, the contents of which are incorporated herein by reference.

### STATEMENT AS TO FEDERALLY SPONSORED RESEARCH

None.

### THE NAMES OF THE PARTIES TO A JOINT RESEARCH AGREEMENT

None.

### SEQUENCE LISTING

None.

### BACKGROUND

Commercial analysis of biological samples containing cells, such as samples containing a microbiome of a subject, can involve providing a sample collection kit to a user, such as a customer. The user deposits the biological sample into a collection container with an aqueous solution, seals the collection container, and returns the sealed container to a facility for analysis. Returning a collection container can involve placing the collection container in a shipping container and providing it to a common carrier for transport of the containers to a facility for processing.

For example, microbiome analysis can involve collection of a feces sample from a user. Collection containers used for such purposes can have a volume capacity of at least six milliliters. Feces can be collected with a swab or an applicator and deposited into the collection container. Swabs or scoops may be discarded after use or included in the collection container with the sample.

Received collection containers are provide to laboratories for processing. Processing can be a multi-step process in which a plurality of aliquots from a collection container are separated into secondary containers. Some of these may be placed into storage, such as a freezer. At least one secondary container is used for analysis. The aliquots for analysis can have a volume appropriate to the processing protocol.

Where the samples contain cells, a step in the processing protocol can include lysing the cells. Cells can be lysed using cell homogenizers, such as bead beating instruments. These instruments use small particles and vigorous agitation to mechanically lyse cells. Containers used with such instruments must have dimensions and be made of materials able to withstand the forces of the instrument.

After cell lysis, biomolecules released from the cells can be further analyzed. This can involve, in the case of nucleic acids, protocols to sequence the nucleic acids present in the sample. Samples can be processed in batches.

Methods to eliminate processing steps in a laboratory facility would improve the efficiency or processing.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows an exemplary container of this disclosure.

FIG. 2 shows another exemplary container of this disclosure.

### SUMMARY

In one aspect provided herein is a method comprising: (a) receiving into a facility one or a plurality of closed containers, each container comprising: (i) a cap comprising an applicator attached thereto and disposed in the container, (ii) solid beads adapted for cell disruption, (iii) an aqueous liquid comprising one or more nucleic acid preservatives, and (iv) a sample comprising cells; (b) subjecting material in the received container to mechanical agitation sufficient to lyse cells, to provide a container comprising a cell homogenate; and (c) providing the cell homogenate to a liquid handling system, wherein the liquid handling system processes the sample, wherein processing comprises isolating nucleic acid from the cell homogenate. In one embodiment subjecting the material in the received container does not comprise transferring some or all of the sample from the received container to another, different, container. In another embodiment the container has a capacity of no more than about 3 ml. in another embodiment the container comprises a bead beating tube compatible with commercially available bead beating instruments. In another embodiment the container comprises a plastic, e.g., polypropylene. In another embodiment the container has a capacity of about 1.5 mL to about 3 mL. in another embodiment the container contains between about 0.5 mL and about 1.5 mL, e.g., about 800 μL of liquid. In another embodiment the container is a screw top container. In another embodiment the applicator has a paddle or spoon-shaped end. In another embodiment the applicator has a stick shape. In another embodiment the applicator has a volume between about 10 μL and 100 μL, e.g., between about 20 μL and about 50 μL. in another embodiment the applicator holds about 5 μg to about 100 μg, e.g., about 30 μg of a liquid. In another embodiment the applicator has a length of about 2 cm to about 4 cm. in another embodiment the cap seals the container through an O-ring. In another embodiment the solid beads comprise zirconium, a glass or stainless steel. In another embodiment the solid beads have a diameter between about 0.1 mm and about 2 mm, e.g., about 0.5 mm or about 1 mm. in another embodiment each container comprises an identifier, e.g., a barcode, that differentiates containers comprising samples from different sources. In another embodiment the nucleic acid preservative comprises an RNA preservative or a DNA preservative. In another embodiment the preservative preserves both RNA and DNA at room temperature for at least two weeks. In another embodiment the sample comprises a fecal sample, saliva sample, a vaginal sample, or a blood sample. In another embodiment the one or a plurality of closed containers received is at least any of 10, 25, 50, 90, 150, or 300 closed containers. In another embodiment the closed containers are received from each of a plurality of different remote locations. In another embodiment receiving comprises receiving, in a single package, a plurality of the closed containers, wherein, (A) at least one of the containers is subjected to the agitation and processing; and (B) at least one of the other containers is received into storage. In another embodiment the method further comprises, before agitating, replacing the cap comprising an applicator with a cap that does not comprise an applicator. In another embodiment agitation comprises bead beating. In another embodiment the cell homogenate is provided to the liquid handling system in the same container. In another embodiment the cell homogenate is provided to the liquid handling system in a different container. In another embodiment processing comprises preparing a DNA library or an RNA library from nucleic acid in the sample. In another embodiment processing comprises: (1) isolating RNA from the sample; and (2) converting the isolated RNA into adapter-tagged cDNA. 30.The method of claim 29, wherein converting comprises digesting DNA in the sample. In another embodiment the method further comprises removing noninformative RNA from the sample. In another embodiment converting comprises reverse transcription of the RNA using a primer comprising an oligonucleotide primer binding site. In another embodiment the process further comprises: (3) combining the adapter tagged cDNA with reagents for performing nucleic acid amplification. In another embodiment the reagents comprise one or more of PCR primers, and a DNA polymerase. In another embodiment processing comprises: (1) isolating DNA from the sample; (2) performing end repair on the isolated DNA; and (3) ligating adapters comprising oligonucleotide primer binding sites to the end repaired DNA to produce adapter tagged DNA. In another embodiment the method further comprises (3) combining the adapter tagged DNA with reagents for performing nucleic acid amplification. In another embodiment the method further comprises (d) preparing, from the isolated nucleic acid, an adapter tagged library; (e) amplifying the adapter tagged library; and (f) sequencing the amplified, adapter tagged library. In another embodiment the liquid handling system operates in an 8×12 tube format. In another embodiment the method further comprises before receiving, transmitting one or a plurality of sample collection kits to each of one or a plurality of locations remote from the facility, wherein each sample collection kit comprises (i) a container, (ii) a cap comprising an applicator attached thereto, (iii) an aqueous solution, and (iv) solid beads adapted for cell disruption. In another embodiment the container has a capacity of no more than about 3 ml. in another embodiment the collection kit is assembled so that the solution and solid beads are contained in the container and the cap seals the container such that the applicator is disposed in the container. In another embodiment the locations are customer locations.

In another aspect provided herein is a closed container comprising: (i) a receptacle containing: (I) an aqueous solution comprising at least one nucleic acid preservative, (II) solid beads adapted for cell disruption, and (III) a sample comprising cells; and (ii) a cap closing the receptacle and comprising an applicator attached thereto and disposed in the receptacle. In one embodiment the container has a capacity of no more than about 3 ml. in another embodiment the cap further comprises a grippable handle.

In another aspect provided herein is a kit comprising: (i) one or a plurality of collection containers, each comprising a receptacle and a cap and, attached to the cap, an applicator, wherein the applicator is configured to fit within the receptacle when closed by the cap; (ii) an aqueous fluid comprising a nucleic acid preservative; and (iii) solid particles having a diameter between about 0.1 mm and about 3.0 mm. in another embodiment the aqueous fluid and solid particles are provided in the collection container sealed by the cap. In another embodiment the kit further comprises a shipping container adapted for holding and shipping the collection container. In another embodiment the cap further comprises a grippable handle.

In another aspect provided herein is a method comprising: (a) providing one or a plurality of closed containers, each container having a capacity of no more than about 3 ml, and comprising: (i) a receptacle; (ii) a cap closing the receptacle and comprising an applicator attached thereto and disposed in the receptacle, (ii) solid beads adapted for cell disruption, and (iii) an aqueous solution; (b) providing a stool sample; and (c) for each of the closed containers: (1) opening the container; (2) collecting a portion of the stool sample with an applicator comprised in the cap; (3) depositing the collected portion of the stool sample into the open container; (4) closing the open receptacle comprising the collected portion of the stool sample with the cap; and (5) shaking the closed container comprising the collected portion of the stool sample. In another embodiment the method further comprises (6) placing the shaken containers into a package; and (7) transmitting the package to a remote location.

In another aspect provided herein is a collection container comprising: (a) a receptacle having an open end; and (b) a cap configured to close the open end, wherein the cap comprises: (i) an elongated applicator extending from a first side of the cap, wherein the elongated applicator is configured to fit within the receptacle when closed by the cap; and (ii) a greppable handle extending from a second side of the cap. In another embodiment the cap further comprises: (iii) a rim that extends beyond the circumference of the open end of the receptacle when the cap closes the receptacle. In another embodiment the handle is configured to be gripped between a human thumb and a finger. In another embodiment the receptacle can accept a volume of between about 0.5 mL and about 1.5 mL of liquid, e.g., about 800 μL in another embodiment the handle has a gripping surface of about 2 cm² to about 4 cm². In another embodiment the handle has a flattened and/or concave shape. In another embodiment the handle has raised gripping features. In another embodiment the cap comprises a closing element comprising a lip that extends outward beyond an edge the open end of the receptacle. In another embodiment the lip extends about 5 mm to about 10 mm beyond the edge.

### DETAILED DESCRIPTION

### I. Introduction

Methods and articles provided herein eliminate aliquoting steps from a process that includes receiving a closed container with a biological sample, aliquoting portions from the container into bead beating container, introducing beads into the bead beading container, lysing cells by mechanical agitation, and subsequent processing, such as nucleic acid isolation.

Methods and articles provided herein include providing collection tubes adapted for use with bead beating instruments to subjects at a location remote from a receiving facility that will receive the tubes. The collection tubes include a cap comprising an applicator, as well as aqueous fluid for dispersing the sample, and solid particles adapted for bead beating, e.g., having diameters of about 0.1 mm to about 3 mm.

Upon receipt of a sample in such a collection container at a processing facility, the aliquoting step is eliminated, and the collection container, optionally with a new cap, can be introduced directly into a bead beating instrument.

After cell lysis, biomolecules in the sample can be analyzed, for example, with a fluid handling system. In one such step, biomolecules of interest are isolated from the sample. For example, nucleic acids can be separated from the remaining homogenate. Isolated biological molecules can be further analyzed. For example, nucleic acids can be prepared into a nucleic acid library and sequenced.

### II. Sample Collection

A. Kit

Users can be provided with kits for sample collection. A kit includes one or, preferably, a plurality of sample collection containers. A kit can include instructions for sample collection. Where the sample is stool, a kit can contain a substrate for collecting stool, such as a cup or a paper sheet for placing on a toilet. A collection container can comprise a receptacle, such as a tube or vial, having an opening for receiving a sample and a cap configured to close or seal the opening in the receptacle.

1. Collection Container/Receptacle

A collection container can be any container compatible for use with a mechanical agitator adapted for cell lysis, e.g., a bead beater instrument. Typically, such receptacles will have a volume capacity of no more than any of 5 mL, 4 mL, 3 mL, 2.5 mL, 2 mL, or 1 mL. Such containers are typically made of a polymer, such as polypropylene. They also can be made from ceramic, glass or metal. They have a thickness sufficient to withstand violent shaking without rupturing. Suitable container types include, for example, the Sarstedt™ screw cap micro tube, tubes from Universal Medical™, OPS Diagnostics™, and DuraTube from SSI bio™. Such tubes typically come in sizes of 1.5 mL, 2.0 mL and 3.0 mL.

Typically, a kit comprises a plurality of collection containers. For example, a kit could comprise two, three or more collection containers. By depositing the biological sample into each of the plurality of collection containers, a user can provide at least one sample for testing and at least one sample for storage.

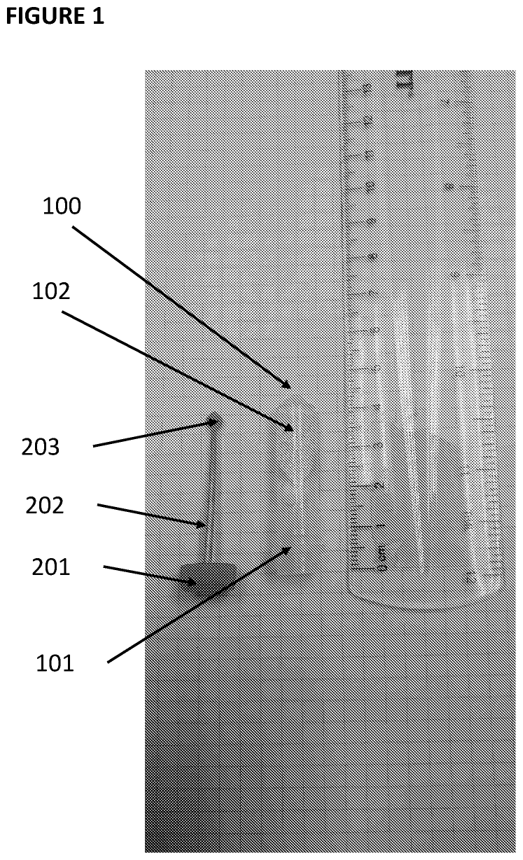

An exemplary collection container is shown in FIG. 1. The figure shows a screwcap tube 100 about 4 cm long having a capacity of about 2.5 mL. The tube has threads 101 for screwing on a cap. Included in the tube are bead beating particles 102. A screw top cap 201 for the tube is also shown. The cap 201 has attached to its inner surface an applicator comprising a stalk 202 and a terminal spoon portion 203. The spoon portion has a capacity of about 30 mg.

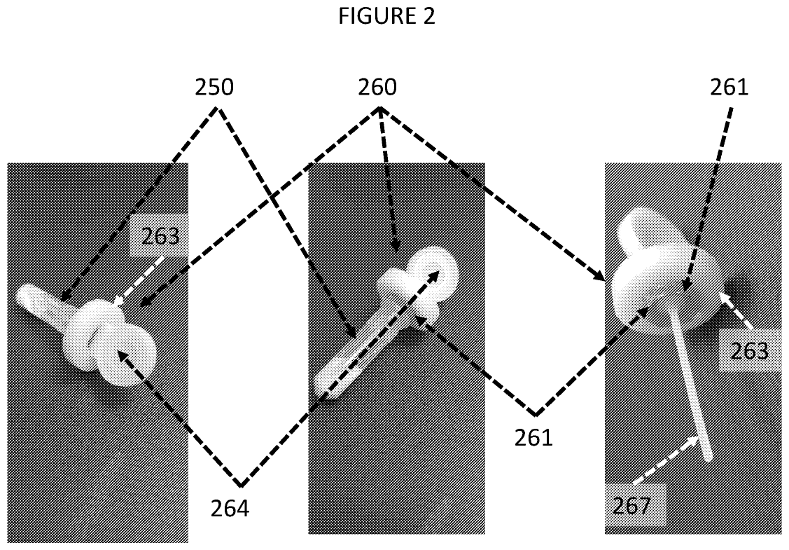

FIG. 2 shows another exemplary embodiment of a collection container 250. The collection container includes receptacle 260 that includes an opening (not shown) that can be fitted with a cap or top 270.

2. Cap

Containers used in the methods of this disclosure comprise a cap to seal the container against leakage of liquid. Typically, the cap is a screw top adapted with threads to mate with threads in the container orifice. Alternatively, the cap can fit in the receptacle opening, and seal with a friction fit, such as a cork. Alternatively, the cap can seal via a luer lock fitting.

The cap can include a ring, such as a gasket, to aid in sealing. Containers also can contain an identifier such as a label or a barcode. The identifier can distinguish containers from different locations or different users or different samples or different collection containers containing sample from the same source from a user. Different containers in the same kit can comprise the same identifier.

The cap also has attached to an inner surface (that is, a surface facing the inside of the container) an applicator or scoop for collecting a portion of a sample and depositing it into the collection container. The applicator has a length such that it is entirely contained within the container when the cap is closed on the container. For example, the applicator may have a length of no more than any of 5 cm, 4 cm, 3 cm, 2 cm or 1 cm. The applicator can be configured as a stick to pick at ordip into the sample. Alternatively, the applicator may have a paddle or a spoon at a distal end. A spoon may have a volume between about 10 microliters and 100 microliters, and may have a capacity of about 10 mg and 100 mg. The paddle or scoop can be connected to the cap through a stalk.

In an embodiment depicted in FIG. 2, top 260 is configured to close or seal the opening of receptacle 250. Closure can be, for example, by a closing element 261 that closes the receptacle by, for example, a pressure fit or a screw mechanism. Top 260 can have, extending from a non-closure side, a handle 264. Handle 264 is configured to be firmly gripped by a human hand and to allow stable manipulation of the cap by user. Such a handle can have a shape and dimensions (e.g., length, width and thickness) that provide a better grip the collection device, in particular during collection and deposition of material, increase the accuracy of collection, help prevent dropping the device during use and/or assist in stabilizing the device during use.

Such manipulation can include, for example, pushing the applicator into a sample, e.g., feces, collecting, e.g., by scooping or picking, sample material, rotating the cap in various dimensions. As such, the handle will typically have sufficient width to gripped between a thumb and finger, and not to rotate without manual impulse. The cap can have dimensions to allow top 260 to be held between a thumb and finger of a user. For example, handle 264 can have a curvilinear shape, such as a circular or ellipsoid shape. It can have a roughly flattened shape, for example, a circular concaved shape. A surface of this flattened shape can have dimensions of, for example, about 70 mm²-about 700 mm², e.g., about 400 mm². For example, the ratio of the length of the handle extending from the top to the width of the handle may be less than 10:1 or less than 5:1. Handle 264 can further comprise gripping features, such as raised edges and/or depressions, to accommodate gripping by the user. On a closure side, top 260 can have a paddle or applicator 267 extending therefrom. Top 260 can further comprise, between handle 247 and applicator 267, a lip or rim 263, that extends outward from the top beyond the edge of the receptacle opening. Rim 263 can function as a guard or shield to protect a user's fingers from touching material collected on a tip of the applicator. The rim 263 can have a side-to-side dimension of between about 10 mm to about 50 mm, e.g., 20 to 30. It can extend beyond an edge of the opening about 2 mm to about 20 mm, e.g., about 5 mm to 15 mm or about 7 mm.

The cap can be made of a single piece of material, e.g., injection-molded plastic. Alternatively, the handle and/or the applicator can be attached to the cap by, for example, an adhesive.

3. Solid Particles

A kit provided to a user also can include solid particles adapted for lysing cells during mechanical agitation, e.g., during bead beating. Particles for bead beating typically are made of zirconium, glass, silica, metal (e.g., stainless steel) or ceramic. They are available in a variety of sizes typically ranging from about 0.1 mm in diameter to about 3 mm in diameter. Typical sizes within this range include, for example 0.1 mm, 0.3 mm, 0.5 mm 1 mm, 2 mm and 3 mm. Beads having a size of 0.1 mm to about 3.0 mm and a total mass within the tube of about 100 mg to about 1000 mg that are useful for lysing bacteria and tissue.

4. Aqueous Fluid

A kit provided to a user can also include an aqueous liquid, e.g., a buffered solution. The aqueous liquid can further contain reagents to inhibit or slow degradation of one or more kinds of nucleic acid, such as DNA or RNA.

As used herein, the term “nucleic acid preservative” refers to a compound or composition that inhibits degradation of nucleic acid. RNA preservatives include, without limitation, formalin, sulfate (e.g., ammonium sulfate), isothiocyanate (e.g., guanidinium isothiocyanate) and urea. Commercially available RNA preservatives include, for example, TRIzol (ThermoFisher), RNAlater (Ambion, Austin, Tex., USA), Allprotect tissue reagent (Qiagen), PAXgene Blood RNA System (PreAnalytiX GmbH, Hombrechtikon), RNA/DNA Shield® (Zymo Research, Irvine, Calif.), and DNAstable (MilliporeSigma, Burlington, Mass.).

5. Sample Collection

A subject providing a sample typically is an animal, e.g., a human, but also can be a nonhuman animal (e.g., bird, reptile or fish), a nonhuman mammal (e.g., a bovine, pig, horse, sheep, goat, dog or cat) or a nonhuman primate (e.g., a monkey or an ape).

The sample to be collected is a biological sample comprising cells. For example, the sample can be feces, blood, saliva, vaginal fluid, or a solid tissue. Feces are particularly useful for the analysis of a subject's gut microbiome. Collection of feces can comprise evacuating the bowels onto a solid support and collecting a portion of the feces into a collection container.

Typically, either or both of the aqueous liquid and/or the solid particles can be included in the collection containers provided to the user. However, they also can be provided in separate containers and added to the sample container by the user. In any case, the user can use the applicator to deposit into the collection container the biological sample comprising cells. Typically, both the aqueous liquid and the solid particles will be included in the collection container provided. In this case, after depositing the sample into the collection container, an individual can close the container such that the applicator, the biological sample, the aqueous solution and the solid particles are sealed in the collection container. An individual can shake the closed container to disperse the sample within the liquid medium.

6. Shipping Container

A kit can further include a shipping container for shipping sample-containing collection containers to a facility that is remote from the user's location. As used herein, the term “remote” when referring to a physical location, refers to a location in another building, e.g., located at least any of 1 mile, 10 miles, 100 miles or 1000 miles away or located in another city, state or country. The shipping container can be any container suitable for shipping through a common carrier or private courier to a recipient. For example, a common carrier can be the United States Postal Service, FedEx or UPS. The shipping container can be, for example, an envelope, a bag, a box or a shipping tube. Such shipping containers can have shipping expenses prepaid.

Accordingly, after the user has deposited the biological sample into one or a plurality of collection containers and sealed the containers such that the applicator, the sample, the aqueous fluid and solid particles are contained within, the collection containers can be transmitted to a facility for processing.

### III. Sample Processing

A. Receipt of Collection Containers

Closed containers received into a facility, such as a collection facility or a laboratory, that contain inside them an applicator, a biological sample, an aqueous liquid and, typically, solid particles can then be processed.

Where a plurality of containers containing samples from the same original biological sample is received from a single remote location, some containers can be designated for processing and some containers can be designated for storage and possible later processing. For example, if three containers are received, one container can be designated for processing and two containers can be designated for storage. Storage can include, for example, freezing samples, e.g., at −80° C.

Typically, a plurality of containers, each containing a different biological sample, e.g., from a different user and/or, from different remote locations, can be processed together, for example, in a batch.

B. Cell Lysis

Processing typically will comprise removing from a container the cap comprising the applicator and replacing it with a cap that does not comprise an applicator. During agitation, an applicator may become dislodged or may disintegrate, fouling the sample.

In any case, the container received into the facility can be the container used for cell lysis. That is, a received collection container containing a sample is deposited into an agitation device, without the need to remove an aliquot of the sample from the collection container into a separate bead beating container (e.g., because collection container is suitable for use with a bead beater). Alternatively, solid particles are not introduced into the container (e.g., because they are already present).

In embodiments of this disclosure, the tube received from the user is the one that goes on the bead beater directly, without intervention by a technician that would otherwise involve opening each tube and aliquoting a sample into the bead beater tube. This further automates the process, eliminating these steps.

Accordingly, this process eliminates the need for technical personnel to transfer user stool specimens to the sample lysis tube(s) from the original collection device.

One or more containers are now subject to mechanical agitation to lyse cells in the sample in the container. A preferred method of mechanical agitation is referred to as “bead beating”. Bead beating involves rapidly shaking a container containing solid particles such that cells in the container are lysed. Bead beating instruments are also referred to as bead-based homogenizers or cell disruptors. They are commercially available from many sources, including, for example, BeadBug™ from Benchmark Scientific™, Bullet Blender™ from Next Advance™ and HT Lysing Bead Mill Homogenizer™ from Ohaus™. Bead beaters can oscillate at around 2000 oscillations per minute.

Mechanical disruption of cells can proceed at, for example, between about five and about fifteen, e.g., eight, shakes per second or over distance of about 3.0 and 10.0, e.g., 6.5, m/s and, for a period of about 30 to about 240, e.g., 90, seconds.

C. Isolation of Nucleic Acids

After cell lysis, samples are further processed by the extraction or isolation of biomolecules in the container, e.g., biomolecules released from lysed cells. Isolated biomolecules typically include nucleic acids such as DNA and/or RNA. Other biomolecules to be isolated can include polypeptides, such as proteins.

Isolation of biomolecules can be performed with a fluid handling robot. Such robots can pipette liquids from tubes, and dispense them into other tubes or plates containing 6-384, e.g., 96, wells, thereby moving aliquots of fluid from one container to another container, e.g., one tube to another tube. This can include, for example, introducing reagents for performing binding or capture events or biochemical reactions. Many fluid handling robots are commercially available. These include, for example, from Tecan, Perkin Elmer, and Hamilton.

Nucleic acids can be isolated from the sample by any means known in the art. Polynucleotides can be isolated from a sample by contacting the sample with a solid support comprising moieties that bind nucleic acids, e.g., a silica surface. For example, the solid support can be a column comprising silica or can comprise paramagnetic carboxylate coated beads or a silica membrane. After capturing nucleic acids in a sample, the beads can be immobilized with a magnet and impurities removed. In another method, nucleic acids can be isolated using cellulose, polyethylene glycol, or phenol/chloroform.

If the target polynucleotide is RNA, the sample can be exposed to an agent that degrades DNA, for example, a DNase. Commercially available DNase preparations include, for example, DNase I (Sigma-Aldrich), Turbo DNA-free (ThermoFisher) or RNase-Free DNase (Qiagen). Also, a Qiagen RNeasy kit can be used to purify RNA.

In another embodiment, a sample comprising DNA and RNA can be exposed to a low pH, for example, pH below pH 5, below pH 4 or below pH 3. At such pH, DNA is more subject to degradation than RNA,

DNA can be isolated with silica, cellulose, or other types of surfaces, e.g., Ampure SPRI beads. Kits for such procedures are commercially available from, e.g., Promega (Madison, WI) or Qiagen (Venlo, Netherlands).

In certain embodiments the target RNA includes RNA anywhere in a sample. In the case of a blood sample, cells in the blood sample can be lysed and all of the RNA isolated. In other embodiments target RNA can include cell free RNA. In such a case, cells will be removed from a sample, e.g. blood, for example by centrifugation and the remaining RNA collected.

Isolation of nucleic acids can further include elimination of non-informative RNA species from the sample. As used herein, the term “non-informative RNA” refers to a form of non-target or non-analyte species of RNA. Non-informative RNA species can include one or more of: human ribosomal RNA (rRNA), human transfer RNA (tRNA), microbial rRNA, and microbial tRNA. Non-informative RNA species can further comprise one or more of the most abundant mRNA species in a sample, for example, hemoglobin and myoglobin in a blood sample. Non-informative RNAs can be removed by contacting the sample with polynucleotide probes that hybridize with the non-informative species and that are attached to solid particles which can be removed from the sample.

D. Further Processing

Isolated nucleic acids can be further processed to produce nucleic acid libraries. Production of nucleic acid libraries typically includes, in the case of RNA, converting RNA into DNA, e.g., by reverse transcription. Adaptors adapted for the DNA sequencing instrument to be used are typically attached to the DNA molecules.

As used herein, the term “adapter-tagged polynucleotide” refers to a polynucleotide comprising a nucleic acid insert flanked on one or both ends by adapter sequences bearing a primer binding site.

As used herein, the term “adapter” refers to a polynucleotide comprising adapter sequences comprising, at least, a primer binding site, e.g., a universal primer binding site or a forward or reverse primer binding site. Adapters also can comprise other elements including, without limitation, a sample barcode, a molecular barcode, a sequencing primer binding site (which may also serve as an amplification primer binding site) or a binding site for binding polynucleotide to platform hardware, such as a flow cell probe binding site. In certain embodiments, adapters can comprise non-complementary ends. These include, for example, “Y-shaped” adapters or adapters which fold back upon themselves to form looped structures. Y-shaped adapters, in particular, can be useful when different strands (“Watson” and “Crick” strands) of a double stranded nucleic acid need to be distinguished. Depending on context, the term “adapter” may also refer to a nucleotide sequence comprising adapter elements.

According to one method, RNA molecules are reverse transcribed into cDNA using a reverse transcriptase. In certain embodiments, primers comprising a degenerate hexamer at their 3′ end hybridize to RNA molecules. The reverse transcriptase extends the primer and can leave a terminal poly-G overhang. In certain embodiments, the primer can also comprise adapter sequences. A template molecule comprising a Poly-C overhang and, optionally, adapter sequences, can be hybridized to the poly-G overhang and used to guide extension to produce an adapter tagged cDNA molecule comprising a cDNA insert flanked by adapter sequences.

Adapter tagged cDNA molecules can be amplified using well-known techniques such as PCR, to produce a library.

Adapters can be attached to DNA molecules through ligation or through primer extension of primers comprising adapter sequences using DNA molecules as a template.

Adapter ligation can involve blunt end ligation or overhang ligation. In blunt end ligation in adapter with a blunt end is ligated to a DNA molecule that also comprises a blunt end. In overhang ligation a DNA molecule with an overhang, such as a “A” overhang or an overhang resulting from restriction endonuclease cleavage, is brought into contact with an adapter molecule comprising a complementary overhang.

Polynucleotides subjected to fragmentation or cell free DNA typically comprise ends with single-stranded overhangs that require end repair before adapter ligation. End repair can be accomplished by, for example, an enzyme such as Klenow which cleaves back 5′ overhangs and fills in 3′ overhangs. The result can be a blunt ended molecule or molecule with a specific overhang.

Alternatively, target polynucleotides can be provided with adapters through a primer extension reaction in which a primer molecule comprises adapter sequences and a sequence that hybridizes to a location in a target polynucleotide. For example, sequence-specific amplification can comprise contacting a DNA sample with primers that hybridize to locations flanking a target sequence. Primers can be extended such that the newly synthesized strand comprises both adapter sequences from the primer and the target sequence upon second strand synthesis in the opposite direction the resulting polynucleotides will comprise a target sequence flanked by adapter sequences. Accordingly, such amplification can comprise multiplex amplification in which a plurality of target sequences is amplified simultaneously.

In the case of DNA, the polynucleotides are typically fragmented. Molecules are end repaired, e.g., by Klenow, to produce either a blunt end or a single nucleotide overhang, e.g., “A”. Adapters comprising blunt ends or having a single “T” overhang can be ligated to the end repaired molecules.

Nucleic acid libraries can be sequenced by any known DNA sequencing methodology. As used herein, the term “high throughput sequencing” includes the simultaneous or near simultaneous sequencing of thousands of nucleic acid molecules. High throughput sequencing is sometimes referred to as “next generation sequencing” or “massively parallel sequencing”. Platforms for high throughput sequencing include, without limitation, massively parallel signature sequencing (MPSS), Polony sequencing, 454 pyrosequencing, Illumina (Solexa) sequencing, SOLiD sequencing, Ion Torrent semiconductor sequencing, DNA nanoball sequencing, Heliscope single molecule sequencing, single molecule real time (SMRT) sequencing (PacBio), nano ball sequencing (Complete Genomics) and nanopore DNA sequencing (e.g., Oxford Nanopore).

Sequencing produces nucleotide sequence information about the nucleic acids sequenced. Such information can be used for any purpose chosen by the operator. For example, where the sample includes nucleic acids from microbes in a microbiome, nucleic acid sequence information can be used to identify relative amounts and types of microorganisms in a sample. If the target analyte includes messenger RNA, sequence information can reveal relative expression levels of genes from various microorganisms. This, in turn, can indicate relative activity of various biochemical pathways.

### EXAMPLES

A user is provided with three screw-top tubes, e.g., a 2 milliliter tube, such as Sarstedt Micro tube 2 ml, PP (manufacturer number 72.608). Each tube has a capacity of about 1.5 ml and contains (1) a top with an applicator attached that holds about 40 mg of material (feces), (2) about 800 ul of buffer with RNA preservative, and (3) zirconium beads for bead beating. The tubes are of a size and thickness to be adapted to and to withstand a bead beater device. The tubes, cap with applicator and, optionally, handle, buffer with RNA preservative, and beads are sterilized and free from contaminating nucleic acids, DNases or RNases. The user uses the applicators to put feces samples in each tube and returns the tubes to a remote collection facility.

An applicator can be molded to a cap with rubber o-ring (similar to a Sarstedt Screw cap; manufacturer number 65.716.729) fitting a Sarstedt Micro tube 2 ml, PP (manufacturer number 72.608). An exemplary small scoop is 43 millimeters in length with the applicator at the end being 5 millimeters in diameter and 2.5 millimeters deep. (See, e.g., FIG. 1). The cap can be prepared by cutting a SuperDosing Static-Free Micro Scoop 30 milligram measuring spoon (X001LBMUH9) to length and affixing it to a Sarstedt Screw cap; manufacturer number 65.716.729.

The cap with attached applicator can be screwed on to a Sarstedt Micro tube 2 ml, PP (manufacturer number 72.608) containing zirconium beads with approximately 800 microliters of RNA preservative and included in a kit for use by a user, e.g. a customer. The customer can unscrew the cap with attached applicator and use the applicator to transfer a small amount of stool specimen to the Sarstedt Micro tube. Users will screw the cap with attached applicator back on to the Sarstedt Micro tube and ship it back to the Viome lab. Each user will receive 3 Sarstedt Micro tubes containing the zirconium beads and RNA preservative with cap with attached applicator and be expected to provide 3 specimens.

Upon specimen arrival at the lab, the cap with attached applicator is be removed and replaced with a Sarstedt Screw cap; manufacturer number 65.716.729. A reason for removing the cap with attached applicator and replacing it with a cap that does not have an attached applicator is to prevent the applicator from being pulverized during the sample lysis procedure, and possibly causing issues with downstream specimen processing.

One Sarstedt Micro tube containing a user's specimen will be transferred to the MP Bio FastPrep-24 5G bead beater for immediate production, while the other 2 Sarstedt Micro tubes will be kept in long-term storage. This will eliminate the need to transfer 1 milliliter of homogenized specimen from a Sarstedt Inc FECES CONTAINER 76/20MM tube to a Sarstedt Micro tube 2 ml, PP containing zirconium beads for specimen lysis and 3 2 milliliter tubes for long term storage/sample reprocessing.

Once lysed, the tube is put into a tray with other tubes and processed by a fluid handler. The fluid handler takes a sample from each tube, and proceeds to process by extracting RNA as a first step in producing an DNA library of RNA sequences for sequencing.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one or more” and “one or a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

All publications, patents, and patent applications mentioned in this specification are herein incorporated by reference to the same extent as if each individual publication, patent, or patent application was specifically and individually indicated to be incorporated by reference.

What is claimed is:

1. A method comprising:
(a) receiving into a facility one or a plurality of closed containers, each container comprising: (i) a cap comprising an applicator attached thereto and disposed in the container, (ii) solid beads adapted for cell disruption, (iii) an aqueous liquid comprising one or more nucleic acid preservatives, and (iv) a sample comprising cells;
(b) subjecting material in the received container to mechanical agitation sufficient to lyse cells, to provide a container comprising a cell homogenate; and
(c) providing the cell homogenate to a liquid handling system, wherein the liquid handling system processes the sample, wherein processing comprises isolating nucleic acid from the cell homogenate.

2. The method of claim 1, wherein subjecting the material in the received container does not comprise transferring some or all of the sample from the received container to another, different, container.

3. The method of claim 1, wherein the container has a capacity of no more than about 3 ml.

4. The method of claim 1, wherein the container comprises a bead beating tube compatible with commercially available bead beating instruments.

5. The method of claim 1, wherein the container comprises a plastic, e.g., polypropylene.

6. The method of claim 1, wherein the container has a capacity of about 1.5 mL to about 3 mL.

7. The method of claim 1, wherein the container contains between about 0.5 mL and about 1.5 mL, e.g., about 800 μL of liquid.

8. The method of claim 1, wherein the container is a screw top container.

9. The method of claim 1, wherein the applicator has a paddle or spoon-shaped end.

10. The method of claim 1, wherein the applicator has a stick shape.

11. The method of claim 1, wherein the applicator has a volume between about 10 μL and 100 μL, e.g., between about 20 μL and about 50 μL.

12. The method of claim 1, wherein the applicator holds about 5 μg to about 100 μg, e.g., about 30 μg of a liquid.

13. The method of claim 1, wherein the applicator has a length of about 2 cm to about 4 cm.

14. The method of claim 1, wherein the cap seals the container through an O-ring.

15. The method of claim 1, wherein the solid beads comprise zirconium, a glass or stainless steel.

16. The method of claim 1, wherein the solid beads have a diameter between about 0.1 mm and about 2 mm, e.g., about 0.5 mm or about 1 mm.

17. The method of claim 1, wherein each container comprises an identifier, e.g., a barcode, that differentiates containers comprising samples from different sources.

18. The method of claim 1, wherein the nucleic acid preservative comprises an RNA preservative or a DNA preservative.

19. The method of claim 1, wherein the preservative preserves both RNA and DNA at room temperature for at least two weeks.

20. The method of claim 1, wherein the sample comprises a fecal sample, saliva sample, a vaginal sample, or a blood sample.

21. The method of claim 1, wherein the one or a plurality of closed containers received is at least any of 10, 25, 50, 90, 150, or 300 closed containers.

22. The method of claim 1, wherein the closed containers are received from each of a plurality of different remote locations.

23. The method of claim 1, wherein receiving comprises receiving, in a single package, a plurality of the closed containers, wherein, (A) at least one of the containers is subjected to the agitation and processing; and (B) at least one of the other containers is received into storage.

24. The method of claim 1, further comprising, before agitating, replacing the cap comprising an applicator with a cap that does not comprise an applicator.

25. The method of claim 1, wherein agitation comprises bead beating.

26. The method of claim 1, wherein the cell homogenate is provided to the liquid handling system in the same container.

27. The method of claim 1, wherein the cell homogenate is provided to the liquid handling system in a different container.

28. The method of claim 1, wherein processing comprises preparing a DNA library or an RNA library from nucleic acid in the sample.

29. The method of claim 1, wherein processing comprises:
(1) isolating RNA from the sample; and
(2) converting the isolated RNA into adapter-tagged cDNA.

30. The method of claim 29, wherein converting comprises digesting DNA in the sample.

31. The method of claim 29, further comprising removing noninformative RNA from the sample.

32. The method of claim 29, wherein converting comprises reverse transcription of the RNA using a primer comprising an oligonucleotide primer binding site.

33. The method of claim 29, wherein the process further comprises:
(3) combining the adapter tagged cDNA with reagents for performing nucleic acid amplification.

34. The method of claim 33, wherein the reagents comprise one or more of PCR primers, and a DNA polymerase.

35. The method of claim 1, wherein processing comprises:
(1) isolating DNA from the sample;
(2) performing end repair on the isolated DNA; and
(3) ligating adapters comprising oligonucleotide primer binding sites to the end repaired DNA to produce adapter tagged DNA.

36. The method of claim 35, further comprising:
(3) combining the adapter tagged DNA with reagents for performing nucleic acid amplification.

37. The method of claim 1, further comprising:
(d) preparing, from the isolated nucleic acid, an adapter tagged library;
(e) amplifying the adapter tagged library; and
(f) sequencing the amplified, adapter tagged library.

38. The method of claim 1, wherein the liquid handling system operates in an 8x12 tube format.

39. The method of claim 1, further comprising, before receiving, transmitting one or a plurality of sample collection kits to each of one or a plurality of locations remote from the facility, wherein each sample collection kit comprises (i) a container, (ii) a cap comprising an applicator attached thereto, (iii) an aqueous solution, and (iv) solid beads adapted for cell disruption.

40. The method of claim 39, wherein the container has a capacity of no more than about 3 ml.

41. The method of claim 39, wherein the collection kit is assembled so that the solution and solid beads are contained in the container and the cap seals the container such that the applicator is disposed in the container.

42. The method of claim 39, wherein the locations are customer locations.

43. A closed container comprising:
(i) a receptacle containing:
(I) an aqueous solution comprising at least one nucleic acid preservative,
(II) solid beads adapted for cell disruption, and
(III) a sample comprising cells; and
(ii) a cap closing the receptacle and comprising an applicator attached thereto and disposed in the receptacle.

44. The container of claim 43, having a capacity of no more than about 3 ml.

45. The container of claim 43, wherein the cap further comprises a grippable handle.

46. A kit comprising:
(i) one or a plurality of collection containers, each comprising a receptacle and a cap and, attached to the cap, an applicator, wherein the applicator is configured to fit within the receptacle when closed by the cap;
(ii) an aqueous fluid comprising a nucleic acid preservative; and
(iii) solid particles having a diameter between about 0.1 mm and about 3.0 mm.

47. The kit of claim 46, wherein the aqueous fluid and solid particles are provided in the collection container sealed by the cap.

48. The kit of claim 46, further comprising a shipping container adapted for holding and shipping the collection container.

49. The kit of claim 46, wherein the cap further comprises a grippable handle.

50. A method comprising:
(a) providing one or a plurality of closed containers, each container having a capacity of no more than about 3 ml, and comprising: (i) a receptacle; (ii) a cap closing the receptacle and comprising an applicator attached thereto and disposed in the receptacle, (ii) solid beads adapted for cell disruption, and (iii) an aqueous solution;
(b) providing a stool sample; and
(c) for each of the closed containers:
(1) opening the container;
(2) collecting a portion of the stool sample with an applicator comprised in the cap;
(3) depositing the collected portion of the stool sample into the open container;
(4) closing the open receptacle comprising the collected portion of the stool sample with the cap; and
(5) shaking the closed container comprising the collected portion of the stool sample.

51. The method of claim 50, further comprising:
(6) placing the shaken containers into a package; and
(7) transmitting the package to a remote location.

52. A collection container comprising:
(a) a receptacle having an open end; and
(b) a cap configured to close the open end, wherein the cap comprises:
(i) an elongated applicator extending from a first side of the cap, wherein the elongated applicator is configured to fit within the receptacle when closed by the cap; and
(ii) a grippable handle extending from a second side of the cap.

53. The collection container of claim 52, wherein the cap further comprises:
(iii) a rim that extends beyond the circumference of the open end of the receptacle when the cap closes the receptacle.

54. The collection container of claim 52, wherein the handle is configured to be gripped between a human thumb and a finger.

55. The collection container of claim 52, wherein the receptacle can accept a volume of between about 0.5 mL and about 1.5 mL of liquid, e.g., about 800 μL

56. The collection container of claim 52, wherein the handle has a gripping surface of about 2 cm² to about 4 cm².

57. The collection container of claim 52, wherein the handle has a flattened and/or concave shape.

58. The collection container of claim 52, wherein the handle has raised gripping features.

59. The collection container of claim 52, wherein the cap comprises a closing element comprising a lip that extends outward beyond an edge the open end of the receptacle.

60. The collection container of claim 59, wherein the lip extends about 5 mm to about 10 mm beyond the edge.
